(19) United States
(12) Patent Application Publication
(10) Pub. No.: US 2009/0022764 A1
(43) Pub. Date: Jan. 22, 2009

(54) ORGANIC COMPOUNDS

(75) Inventors: Georg Frater, Winterthur (CH); Rita Cannas, Duebendorf (CH); Benjamin J. Costa, Nutley, NJ (US); Jutta Hotz, Zurich (CH)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 11/573,969

(22) PCT Filed: Aug. 26, 2005
(86) PCT No.: PCT/CH2005/000500

§ 371 Date: Apr. 23, 2007

(30) Foreign Application Priority Data
Aug. 31, 2004 (GB) 0419268.8

(51) Int. Cl. A61K 8/06; C11D 3/37

(52) U.S. Cl. 424/401; 510/527

(57) ABSTRACT

The present invention refers to a process for the production of controlled-release aqueous compositions comprising an active ingredient releasably contained within polymeric particles. The polymeric particles are obtainable by ring-opening metathesis polymerisation of a monomer of the general formula (I)

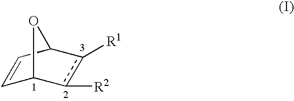

wherein R¹ and R² have the same meaning as given in the specification, or a mixture thereof, in an aqueous phase in the presence of a ruthenium catalyst, an emulsifier, and an active ingredient.

The present invention refers to a process for the production of controlled-release aqueous compositions comprising an active ingredient releasably contained within polymeric particles.

There is a constant demand to provide new technologies to protect active ingredients, such as fragrances, antioxidants, antibacterials and UV-filters, during the manufacture and storage of aqueous household and personal care products, such as detergents and conditioners, cleansing products and cosmetic products, comprising said agents, which are also capable of releasing the active slowly upon usage.

A principal strategy which is currently employed in imparting odors to consumer products is the admixing of the fragrance directly into the product. There are several drawbacks to this strategy. The fragrance can be too volatile, resulting in fragrance loss during manufacturing, storage, and use. Many fragrances are also unstable over time. This again results in loss during storage.

It is also well known that the release of an active ingredient from a composition can be controlled by trapping the active ingredient within polymeric particles. Such particles are obtainable either by forming polymeric particles and then absorbing the active ingredient or by incorporation of the active ingredient during the formation of the particles. For example, DE 967 860 describes a process for the encapsulation of perfume by polymerisation or poly-condensation of, for example, urea, acrylonitrile or vinyl acetate, to provide solid particles comprising up to 40% by weight of perfume. The perfume is added either before or after the polymerisation process has already been started. EP 0617051 discloses polymeric compositions obtained by emulsion polymerisation of unsaturated momomers, namely alkyl(meth)acrylates, in the presence of a fragrance. The particles are formed by oil-in-water emulsion polymerisation of the water-insoluble monomer in which the active ingredient, such as an agricultural semiochemical, is dissolved. WO 01/79303 discloses polymeric nanoparticles including olfactive components obtainable by a semicontinuous polymerisation. Styrene and acrylic acid derivatives as monomer components in the presence or absence of a cross-linking agent are exemplified.

Surprisingly, we found that heteropolycyclic alkenes of formula (I)

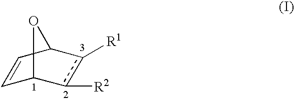

wherein
R¹ and R² are independently methoxymethyl or —CO₂Me; or
R¹ and R² form together a divalent radical —C(O)N(CH₃)C(O)—which forms together with the carbon atoms to which they are attached a 5-membered heterocyclic ring; and
the bond between C2 and C3 is a single bond; or
the bond between C2 and C3 together with the dotted line represents a double bond; are suitable for the production of a delivery system for controlled release of active ingredients.

Whereas the polymerisation of heteropolycyclic alkenes by ring-opening metathesis polymerisation (ROMP) is known in general (W. Kames Feast et al., Journal of Molecular Catalysis, 65 (1991) 63-72), the encapsulation of active ingredients during the formation of polymer particles from these monomers has not been described. The ring-opening methathesis polymerisation is a polymerisation method which transforms cyclic unsaturated monomers into a linear unsaturated polymer in the presence of a catalyst.

Accordingly, the present invention refers in one aspect to an aqueous emulsion comprising polymer particles containing an active ingredient, wherein the polymer with a Tg between 15° C. and 150 ° C. is obtainable by ring-opening metathesis polymerisation of a monomer of the general formula (I)

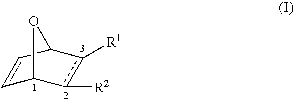

wherein
R¹ and R² are independently methoxymethyl or —CO₂Me; or
R¹ and R² taken together is a divalent radical —C(O)N(CH₃)C(O)—which forms together with the carbon atoms to which they are attached a 5-membered heterocyclic ring; and
the bond between C2 and C3 is a single bond; or
the bond between C2 and C3 together with the dotted line represents a double bond;
or a mixture thereof,
in an aqueous phase in the presence of a ruthenium catalyst, an emulsifier, and an active ingredient.

Ruthenium catalysts particularly useful for the present invention are selected from ruthenium trichloride (RuCl₃×3 H₂O), available e.g. from Aldrich Chemical Co. Ltd, and RuCl₂(PCy₃)₃CHPh, known as Grubbs I catalyst. Particularly preferred is the use of ruthenium trichloride because it is easy to handle, water-soluble and cheap.

The polymer that is used to form the polymer particles according to this invention may be homopolymers or copolymers. The copolymers may comprise two or more monomers.

Polymer particles having a particle size between 50 nm to 100 μm, preferably between 100 nm and 1 μm, most preferred between 150 nm and 400nm are preferred. The particle size may be measured for example by transmission electronic microscopy (TEM). This method is also well suited to determine the distribution of residual catalyst on the surface and inside the particle. Surprisingly it has been found that the catalyst is uniformly distributed on the surface of the particles and mono-dispersed particles are formed.

Several parameters, such as the choice and amount of the active ingredient, the monomer and/or the emulsifier may influence the nature of the final polymer and therefore the Tg (glass transition temperature). The polymer should have a Tg between 15° C. and 150° C., preferably between 35° C. and 130° C., most preferably between 40° C. and 100° C., which may be determined by standard methods well known in the art.

The active ingredients suitable for encapsulation according to the present invention may be any ingredients that are desired in encapsulated form, for example, insect repellents, antibacterials, antioxidants, UV-filters and fragrances. The amount of the active ingredient suitable for encapsulation mainly depends on the desirable effect of the end-product. For example, if the active ingredient is a fragrance, an amount up to 15% by weight, preferably about 1 to 9% by weight, based on the emulsion, is suitable. Suitable fragrances may be selected from the extensive range of natural products and synthetic molecules currently available, such as essential oils, alcohols, aldehydes and ketones, ethers and acetals, esters and lactones, macrocylces and heterocycles. Suitable insect repellents include but are not limited to N,N-diethyl-m-toluamide (DEET), N-ethyl-p-menthane-3-carboxamide (WS-3), N,N-diethyl-benzamide, menthyl 2-pyrrolidone-5-carboxylate, N-aryl and N-cycloalkyl neoalkanamides, N-lower alkyl neoalkanamides, nepetalactone, and natural oils known for their insect repellent characteristics. Examples for such oils include, without limiting, citronella oil, catnip oil, eucalyptus oil, cypress oil, galbanum oil, tolu and Peru balsams. Suitable UV-filters include but are not limited to p-methoxy cinnamic acid iso-amyl ester, cinnamates, salicylic acid esters, 4-amino benzoic acid derivatives and sulphonic acid derivatives of benzophenone and 3-benzylidene camphor. Suitable antibacterials include but are not limited to triclosan, farnesol, monolaurin-glycerol and other glycerol esters or glycerol mono-ethers. Suitable antioxidants include but are not limited to L-ascorbic acid and its salts, ascorbyl palmitate, ascorbyl stearate, erythorbic acid and its salts, tocopherol, alkyl gallate, butylated hydroxyanisole (BHA), butylated hydroxytoluene (BHT), and lecithins.

Preferably the active ingredient or mixtures thereof is a liquid having lipophilic properties. Surprisingly it has been found that the polymerisation time decreases in the presence of fragrance ingredients. Thus, fragrances are particularly preferred for encapsulation according to the present invention.

The choice of a suitable emulsifier is less critical in the polymerisation process of the present invention. Anionic, cationic and non-ionic emulsifiers are suitable, however anionic and cationic emulsifiers are preferred. However, surprisingly it was found that, due to the choice and concentration of the emulsifier, the release kinetics of the polymer particles in the end-product, e.g. in a fabric conditioner when contacted with the fabric, can be influenced. The emulsifier is preferably used in an amount of 1 to 8% by weight based on the aqueous phase, more preferred in amount between 4 and 7% by weight. It is well known in the art that the more emulsifier is used the smaller the particle size of the polymer particles is.

The following list comprises examples of emulsifiers suitable for the preparation of polymer particles according to the present invention:
cationic emulsifiers, e.g. ricinoylamidopropyltrimethyl-ammoniummetho sulfate, cocopentylethoxymethyl-ammoniummetho sulfate, cocobis(2-hydroxyethyl) methylammonium chloride, cetyltrimethylammonium bromide, glyceryl stearate and stearamidoethyl diethylamine, or mixtures thereof. Most preferred cationic emulsifier include ricinoylamidopropyltrimethyl-ammoniummetho sulfate available from Goldschmidt under the trade name Rewoquat RTM50.
non-ionic emulsifiers, e.g. ethoxylated linear fatty alcohols, such as C₁₂-C₁₄ fatty alcohols ethoxylated with ethylene oxide, ethylene oxide/propylene oxide block copolymers, e.g. available from Uniqema under the trade name Synperonic™, sorbitan stearate, polysorbate, and stearate, or mixtures thereof.
anionic emulsifiers, e.g. sodium dodecyl sulfate, ammoniumnonoxynol-sulfate, e.g. available from Rhodia under the trade name Abex EP-227, and glyceryl stearate, or mixtures thereof. In general, the polymerisation is performed by admixing the monomer of formula (1), the active ingredient, emulsifier and water at room temperature or elevated temperature to obtain an emulsion, followed by initiation of the polymerisation reaction by addition of the ruthenium catalyst. The polymerisation reaction is preferably performed under elevated temperature up to about 70° C., more preferably at about 60° C. or lower. Depending on the reactivity of the monomer and the catalyst, it may take between a few hours, e.g. two to three hours, up to several days until the polymerisation reaction is completed. Whether the polymerisation process is completed or not may be analysed by measuring the monomer concentration by methods known to the persons skilled in the art. The polymerisation reaction is completed when the monomer concentration of the aqueous phase is stable over a longer period. Preferably the concentration does not decrease within 48 hours, more preferably within 24 hours, most preferred within 8 hours. The polymer particles contained in the emulsion may optionally washed with a liquid phase in which the catalyst is soluble, for example, water, water/ethanol mixture or water/acetone mixture, or with a liquid phase comprising a ruthenium complexing agent, such as CH₃CN, EDTA, or 2,2′-bipyridyl.

Alternatively, the polymerisation is performed by admixing the monomer with the active ingredient, and separately the water with the emulsifier. The premixed monomer/active ingredient mixture is then added with stirring to the vessel comprising the water/emulsifier mixture. While continuing stirring, the catalyst is added and the resulting mixture is heated. It has been found that a more stable emulsion is obtained when the active ingredient is added to the monomer first before admixing with the water and emulsifier.

Accordingly, the present invention relates in a further aspect to a method of preparing polymer particles comprising the steps of
a) admixing a monomer of the general formula (I) or a mixture of such monomers, an active ingredient, water and an emulsifier at room temperature;
b) adding the ruthenium catalyst to the mixture resulting from step a) and
c) heating the mixture resulting from step b); and
d) optionally washing the polymer particles after the polymerisation reaction is completed.

The aqueous emulsion comprising the polymer particles may be used in any liquid home care and fabric care product, for example, detergents, fabric conditioners, rinsing conditioners for fabrics, hard surface cleaners, cosmetic products, such as body cleansing compositions and rinse-off hair conditioners, or spray applications, e.g. on carpets and furniture. The emulsion is especially suitable for applications in cosmetics, hair and fabric conditioners. A significant improved deposition of the fragrance on the substrate, e.g. fabric or hair, has been found when the polymer particles containing the fragrance instead of the free fragrances are added to a conditioner.

If used for example in cosmetic applications such as powders, the polymer particles are preferably used in dried form, obtainable by drying of the aqueous polymer emulsion by methods known to the skilled person. In general such drying methods are preferred wherein the drying temperature is 70° C. or lower.

In the case of an encapsulated fragrance, a significant improvement of deposition means a measurable increase of the fragrance concentration on the substrate measured by solvent or thermal extraction.

The invention is now further described with reference to the following non-limiting examples.

### EXAMPLE 1

Polymerization of 4-methyl-10-oxa-4-aza-tricyclo[5.2.1.0^{2,6}]dec-8-ene-3,5-dione

4-methyl-10-oxa-4-aza-tricyclo[5.2.1.0^{2,6}]dec-8-ene-3,5-dione was prepared by Diels-Alder reaction of furan and maleic imide (according to H. Kwart et al. J. Am. Chem. Soc. 1952, 74, 3094) followed by methylation (according to P. Camps et al., Chem. Be. 1995, 127, 1933).

20 g of 4-Methyl-10-oxa-4-aza-tricyclo[5.2.1.0^{2,6}]dec-8-ene-3,5-dione, 6.68 g of Abex2030, 8.0 g of fragrance composition and 80 ml of water were mixed with the Ultraturax for ca 5 min to obtain an emulsion. 440 mg (0.015 eq.) of RuCl₃×3H₂O were added and the reaction was mechanically stirred and heated at 60° C. for 17 hours.

### Tg=137° C.;

medium particle size: 210 nm, measured by transmission electronic microscopy (TEM);

The fragrance composition used in the examples contains the following fragrance ingredients: iso-Amylacetate (3-methyl-1-butyl acetate), Eucalyptol, Dimethyloctenon, Cyclal C, Linalool, Aldehyde C12, Viridine, Terpineol, Benzylacetate, Irisone Alpha, Verdylacetate, Phenylethanol, Diphenyloxid, Prunolide and Lilial.

### EXAMPLE 2

Polymerization of 7-oxa-bicyclo[2.2.1]hepta-2,5-diene-2,3-dicarboxylic acid dimethyl ester 3 g of 7-Oxa-bicyclo[2.2.1]hepta-2,5-diene-2,3-dicarboxylic acid dimethyl ester (prepared according to Eberbach, W.; Perroud-Arguelles, M.; Achenbach, H.; Druckrey, E.; Prinzbach, H. Helvetica Chim. Acta 1971, 54, 2579), 0.5 g of Rewoquat RTM50, 0.6 g of the fragrance composition as describe in Example 1 and 10 ml of water were mixed with the Ultraturax for ca 5 min to obtain an emulsion. 56 mg (0.015 eq.) of RuCl₃×3H₂O were added and the reaction was mechanically stirred and heated at 60° C. for 4 hours.

Tg=53° C.

### EXAMPLE 3

Copolymerization of 4-methyl-10-oxa-4-aza-tricyclo[5.2.1.0^{2,6}]dec-8-ene-3,5-dione and 5,6-Bis-methoxymethyl-7-oxa-bicyclo[2.2.1]hepta-2-ene

5,6-Bis-methoxymethyl-7-oxa-bicyclo[2.2.1 ]hepta-2-ene was prepared by Diels-Alder reaction between furan and maleic anhydride (according to H. Stockmann, J. Org. Chem. 1961, 26, 2025), reduction of the resulting product (according to J. Das et al, J. Med. Chem. 1988, 31, 930), followed by methylation of the diol (according to J. T. Manka et al, J. Org. Chem. 2000, 65, 5202).

7 g of 4-Methyl-10-oxa-4-aza-tricyclo[5.2.1.0^{2,6}]dec-8-ene-3,5-dione, 3 g of 5,6-bis-methoxy-methyl-7-oxa-bicyclo[2.2.1]hepta-2-ene, 3.33 g of Abex™2030, 4.0 g of the fragrance composition as describe in Example 1 and 40 ml of water were mixed with the Ultraturax for 5 min to obtain an emulsion. 217 mg (0.015 eq.) of RuCl₃×3H₂O were added and the reaction was mechanically stirred and heated at 60° C. for 17 hours. Tg=70° C.

### EXAMPLE 4

Polymerization of 4-methyl-10-oxa-4-aza-tricyclo[5.2.1.0^{2,6}]dec-8-ene-3,5-dione

60 ml of water and 2.02 g of Synperonic™ PEF127 in a beaker and then the premix of the monomer/fragrance composition (20 g/6 g) is portion wise added and mixed with the Ultraturax for about 5 min to obtain an emulsion. 438 mg (0.015 eq.) of RuCl₃×3H₂O were added and the reaction was mechanically stirred and heated at 60° C. for 8 hours.

### Tg=127° C.

### EXAMPLE 5

### Recovery of Active Ingredients

The water of the emulsions prepared according to the Examples 1 and 4 was separated from the polymer particles using an ultracentrifuge. The water phase was extracted with CH₂Cl₂ where by the polymer particles were dissolved in CH₂Cl₂. Both, the organic phases separated from water and the polymer particles were analysed by GC. The results are shown in Table 1.

TABLE 1
Recovery of the active ingredients from the emulsion comprising
the polymer particles

| | recovery | | |
|---|---|---|---|
| Emulsifier | overall | polymer particles | water phase |
| Abex ™ 2020 (Ex. 1) | 67% | 42% | 25% |
| Synperonic ™ PEF127 (Ex. 4) | 75% | 60% | 15% |

The water phase contains partially the most hydrophilic compounds. An overall recovery of up to 75% has been found for the test fragrance composition used. Some compounds, such as Viridine, are unstable under the polymerization conditions used and it is believed, without bound by theory, that certain fragrance ingredients may be chemical bound to the polymer and accordingly are not longer detectable per se.

### EXAMPLE 6

### Rinse Test (Example 1)

Samples of a fabric rinse conditioner (concentrated or diluted) were prepared containing 0.95% of fragrance: one in free form (blank) and one encapsulated in the polymer prepared according to Example 2. A rinse test was carried out using cotton towels (20×20 cm) for each sample. 1.43 g of softener was used comprising the free fragrance and 1.62 g of softener comprising the encapsulated fragrance. The towels were shaken for 10 min in one litre cold water comprising the fabric rinse conditioner, centrifuged for 30 sec and then let drying. A smell test, using a Labeled Magnitude Scale (LMS) described for example by B. G. Green et al. in Chem. Senses 21:323-334, 1996, was performed by a panel of 9 experts after one and five days. The average results are shown in Table 2.

TABLE 2
Smell test using diluted/concentrated cationic fabric rinse conditioner

| | after 1 day | after 5 days |
|---|---|---|
| diluted fabric rinse conditioner | | |
| with free fragrance | 2.4 | 1.0 |
| with encapsulated fragrance | 3.0 | 1.9 |
| concentrated fabric rinse conditioner | | |
| with free fragrance | 1.3 | 0.6 |
| with encapsulated fragrance | 2.9 | 2.3 |

0 = not perceivable, 1 = very weak, 2 = weak, 3 = medium, 4 = strong, 5 = very strong.

### EXAMPLE 7

### Stability Test

The stability of fabric rinse conditioners comprising fragrance in an encapsulated form of Example 6 has been analyzed. All samples were stable for at least one month at 4, 20 and 40° C. storage temperature.

1. An aqueous emulsion comprising polymer particles containing an active ingredient, wherein the polymer with a Tg between 15° C. and 150° C. is obtainable by ring-opening metathesis polymerisation of a monomer of the general formula (I) 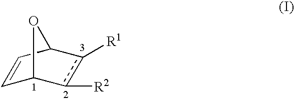
wherein
R¹ and R² are independently methoxymethyl or —CO₂Me; or
R¹ and R² form together a divalent radical —C(O)N(CH₃)C(O)—which forms together with the carbon atoms to which they are attached a 5-membered heterocyclic ring; and
the bond between C2 and C3 is a single bond; or
the bond between C2 and C3 together with the dotted line represents a double bond;
or a mixture thereof,
in an aqueous phase in the presence of a ruthenium catalyst, an emulsifier, and an active ingredient.

2. An aqueous emulsion comprising polymer particles according to claim 1 wherein the active ingredient is selected from the group consisting of insect repellents, antibacterials, antioxidants, UV-filters and fragrances.

3. An aqueous emulsion comprising polymer particles according to claim 1 wherein the polymer particles containing up to 15% by weight of a fragrance as active ingredient.

4. A method of preparing polymer particles comprising the steps of:
a. admixing a monomer of the general genera formula (I) or a mixture thereof, 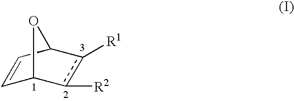
wherein:
R¹ and R² are independently methoxymethyl or —CO₂Me; or
R¹ and R² form together a divalent radical —C(O)N(CH₃)C(O)—which forms together with the carbon atoms to which they are attached a 5-membered heterocyclic ring; and
the bond between C2 and C3 is a single bond; or
the bond between C2 and C3 together with the dotted line represents a double bond;
an active ingredient, water and an emulsifier at room temperature;
b. adding an ruthenium catalyst to the mixture resulting from step a);
c heating the mixture resulting from step b); and
d. optionally washing the polymer particles after the polymerisation reaction is completed.

5. A method according to claim 4 comprising a further step of drying the polymer particles after the polymerisation reaction of step a. to c. is completed.

6. A method according to claim 4 wherein the active ingredient is selected from the group consisting of insect repellents, antibacterials, antioxidants, UV-filters and fragrances.

7. A home care, fabric care or cosmetic product comprising polymer particles containing an active ingredient obtained according to claim 1.
